(19) United States
(12) Patent Application Publication
(10) Pub. No.: US 2020/0397415 A1
(43) Pub. Date: Dec. 24, 2020

(54) SYSTEMS AND METHODS FOR STOOL SAMPLE COLLECTION

(71) Applicant: BiomeSense, Inc., Chicago, IL (US)

(72) Inventors: Catherine Yunis, Los Angeles, CA (US); Dylan Nichols, Chicago, IL (US); Marc Auger, New York, NY (US)

(21) Appl. No.: 16/902,795

(22) Filed: Jun. 16, 2020

Related U.S. Application Data
(60) Provisional application No. 62/864,266, filed on Jun. 20, 2019.

(51) Int. Cl. A61B 10/00; A61B 5/00

(52) U.S. Cl. CPC A61B 10/0038; A61B 2562/06; A61B 2560/04; A61B 5/6891

(57) ABSTRACT

Methods and systems directed to collecting and processing stool samples are disclosed herein. A stool sample may be collected from a subject using a stool collector, which may comprise one or more catchment units. The stool sample may then be subjected to further processing and/or analysis to determine one or more parameters of the stool sample.

### CROSS-REFERENCE

This application claims the benefit of U.S. Provisional Patent Application No. 62/864,266, filed Jun. 20, 2019, which is incorporated by reference herein in its entirety.

### BACKGROUND

A sample may be collected for various purposes, such as identification of a property of the sample. The sample may be a biological sample or biology-derived sample. Biological samples may be processed, such as for disease detection and diagnosis, identification of contaminants. Various approaches for processing samples may be performed, such as polymerase chain reaction (PCR) and sequencing.

Biological samples may be collected using a variety of approaches, such as physical capture. Devices or systems may be employed to capture or collect a biological sample that enables further analysis of the biological sample.

Biological samples may be subjected to various processes, such as chemical or physical processes. Samples may be subjected to heating or cooling, chemical reactions, mechanical reactions, such as to yield a sample or species that can processed qualitatively or quantitatively.

### SUMMARY

In an aspect, disclosed herein is a method for processing a stool sample, comprising: (a) using a stool collector comprising a plurality of sensors to collect a stool sample of a subject from a location within a toilet bowl, wherein the stool collector is coupled to a fluid flow path separate from the stool collector; (b) directing the stool sample along the fluid flow path to process the stool sample to yield a processed stool sample; and (c) collecting the processed stool sample.

In some embodiments, a sensor of the plurality of sensors comprises a non-optical sensor. In some embodiments, the sensor is a weight sensor. In some embodiments, the sensor is a water level sensor. In some embodiments, the sensor is a motion sensor. In some embodiments, the collection of the stool sample by the stool collector is initiated automatically. In some embodiments, the stool collector automatically turns on to collect the stool sample and automatically turns off subsequent to collection of the stool sample. In some embodiments, the stool collector comprises a source of reagents that is coupled to the fluid flow path. In some embodiments, the stool collector is reusable. In some embodiments, the stool collector is retractable. In some embodiments, the stool collector deposits the stool sample into a sample homogenization chamber. In some embodiments, the processing occurs within the location of the toilet bowl. In some embodiments, the method further comprises, following (c), automatically cleaning at least a portion of the stool collector. In some embodiments, the stool collector comprises catchment fingers that collect the stool sample. In some embodiments, the stool collector comprises electronics coupled to a communication device. In some embodiments, the fluid flow path is directed out of the toilet bowl. In some embodiments, the stool collector is attached to a toilet seat coupled to the toilet bowl. In some embodiments, the stool collector is attached to the toilet bowl.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example workflow for processing a stool sample.

FIG. 2 schematically illustrates an example stool collector. FIG. 2A shows a stool collector coupled to a toilet seat. FIG. 2B shows the underside of the toilet seat and the coupled stool collector. FIG. 2C shows an enlarged view of the underside of the toilet seat and the stool collector. FIG. 2D shows another view of the example stool collector.

FIG. 3 schematically shows an example catchment unit.

FIG. 4 schematically shows an example geometry of catchment fingers of a catchment unit.

FIG. 5 schematically shows an example valve that may be used in one or more methods or systems described herein.

FIG. 6 shows an example of an assembled stool collector coupled to a toilet seat.

FIG. 7 shows an example of a source of reagents that is coupled to a fluid flow path.

FIG. 8 schematically shows an example process flow diagram of the source of reagents coupled to the stool collector and collection of the stool sample.

FIG. 9 schematically shows example data of sterilization of the stool collector. FIG. 9A shows a table of the measured concentration of DNA of various samples. FIG. 9B shows a table of the measured concentration of DNA and amplification of DNA of various samples.

FIG. 10 schematically shows another example of a stool collector.

FIG. 11 schematically shows another example of a stool collector. FIG. 11A shows the stool collector in a closed configuration. FIG. 11B shows the stool collector in an open configuration, e.g., during sample collection.

FIG. 12 schematically shows another example of a stool collector. FIG. 12A shows the stool collector in a closed configuration. FIG. 12B shows the stool collector in an open configuration, e.g., during sample collection.

FIG. 13 schematically shows another example of a stool collector comprising a gasket. FIG. 13A shows the stool collector in a closed configuration. FIG. 13B shows the stool collector in an open configuration, e.g., during sample collection.

FIG. 14 schematically shows another example of a stool collector. FIG. 14A shows the stool collector in a closed configuration. FIG. 14B shows the stool collector in an open configuration, e.g., during sample collection.

FIG. 15 schematically shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer), or a pre-disposition to the disease, an infection, and/or an individual that is in need of therapy or suspected of needing therapy. A subject can be a patient. A subject can be a microorganism or microbe (e.g., bacteria, fungi, archaea, viruses).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA, complementary DNA, etc.). Sequencing can be performed by various approaches, such as, without limitation, a sequencing system by Illumina®, Pacific Biosciences (PacBio®), Oxford Nanopore®, or Life Technologies (Ion Torrent®). Alternatively or in addition to, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sample,” as used herein, generally refers to a biological sample of a subject. The biological sample may comprise any number of macromolecules, for example, cellular macromolecules. The sample may be a stool sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from (e.g., processed from) another sample. The sample may be a stool sample or derived from a stool sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

### Systems and Methods for Stool Sample Collection

In an aspect, the present disclosure provides systems and methods for the collection, compartmentalization, and/or processing of one or more samples. The sample may be collected from a subject, e.g., a human, and may comprise a stool sample, or derivative thereof. A method for processing a stool sample may comprise using a stool collector comprising a plurality of sensors to collect a stool sample of a subject from a location within a toilet bowl. The stool collector may be coupled to a fluid flow path separate from the stool collector. The method may further comprise directing the stool sample along the fluid flow path to process the stool sample to yield a processed stool sample and collecting the processed stool sample.

In some instances, the stool collector may comprise automated sample collection. Methods and systems described herein may allow for stool collection that does not require a subject to interact with the stool sample.

The stool collector may be configured to couple to a toilet. In some instances, the stool collector may comprise a housing that is configured to couple to a toilet. The stool collector may be attached to a toilet seat, which may be coupled to the toilet bowl. In some cases, the stool collector is attached to the toilet bowl. The stool collector may be removably coupled to the toilet. The stool collector may be mechanically coupled to the toilet using one or more fastening mechanisms. For example, the stool collector may comprise threads (e.g., screw threads, internal threads) and the toilet may comprise complementary threads that may engage with the threads of the stool collector. Alternatively or in addition to, the stool collector may comprise snap-fit joints (e.g., cantilever snap fits, annular snap fits, etc.) that allow for interlocking of the stool collector to the toilet. Other interlocking or form-fitting pairs may be used to secure the stool collector to the toilet, e.g., hook and loop, latches, snap-ons, buttons, nuts and bolts, screws, magnets, etc. Alternatively or in addition to, the stool collector may comprise components that allow for fitting into the toilet, e.g., via an interference fit, force fit, shrink fit, location fit, etc. Other fastening mechanisms are possible, such as form-fitting pairs, hooks and loops, latches, threads, screws, staples, clips, clamps, prongs, rings, brads, rubber bands, rivets, grommets, pins, ties, snaps, Velcro, vacuum, seals, or a combination thereof. The stool collector may be adhered to the toilet (e.g., using adhesive tape or glue).

The stool collector may be retractable or comprise a catchment unit that is retractable. In some instances, the stool collector comprises a chamber that houses the catchment unit. The catchment unit may comprise one or more catchment arms or rods. The catchment arm may be configured to extend towards the toilet bowl during sample collection. In some instances, the catchment arm may be configured to retract towards a chamber (e.g., homogenization chamber), which may be the same chamber or a different chamber than the chamber that houses the catchment unit. The extension and/or retraction of the catchment unit or arm may be initiated automatically. In some cases, the extension may be initiated using one or more signals from a sensor, as described elsewhere herein. Alternatively or in addition to, the retraction of the catchment unit may be initiated using a signal from a sensor of the stool collector. During or following stool sample collection, one or more sensors may detect completion or sufficiency of the sample collection (e.g., via a weight sensor, optical sensor, motion sensor, etc.) and retraction may be initiated. In some cases, a timer may be integrated with the stool collector, such that the catchment unit retracts after a given period.

The stool collector, or portion thereof (e.g., the catchment unit) may comprise one or more gears. In some cases, the extension of the catchment unit may be performed using one or more gears. The extension of the catchment unit may also be performed using a variety of mechanisms, e.g., mechanical, electrical, or other approaches. In some examples, the stool collector may comprise a plurality of gears that can interface with a motor. In some cases, the extension and retraction of the catchment unit may be driven by a stepper motor or brushed or brushless DC or AC driven motor that interacts with a set of gears and a guide screw. Altering the motor speed via electrical modulation, or mechanically switching the gear set may change the speed of actuation. In some cases, an electric linear actuator or servo motor may be used to extend and retract the catchment unit.

In some instances, gears may also be used to couple with another part of the stool collector. In some cases, a gear may be configured to couple to a mechanical valve (e.g., luer valve). In some instances, gears may be used to couple a motor to a valve and/or to a source of reagents. A plurality of gears of different sizes may be used. The gears may be secured to a custom-built base, which may secure to a portion of the toilet or the stool collector. The custom-built base may be built using a variety of approaches, e.g., machining, milling, 3D printing, injection molding, etc.

One or more motors may be coupled to one or more parts of the stool collector. The stool collector may comprise any suitable type of motor, e.g., an electric motor, a manual motor, an air motor, etc. In some instances, the motors may be coupled to one or more gears and/or one or more pumps. One or more motors may be used for a variety of purposes, including but not limited to: opening and/or closing or the homogenization chamber, extension and/or retraction of the catchment unit, homogenization of the stool sample, pumping a fluid or reagent into the homogenization chamber, directing the stool sample or processed stool sample (e.g., homogenized stool sample) away from the toilet and/or to a collection unit.

The stool collector may comprise a catchment unit. The catchment unit may be used to capture and/or collect the stool sample. The catchment unit may comprise any useful geometry. For instance, the catchment may comprise a plurality of catchment appendages (e.g., “fingers”) that may be configured to collect the stool sample. Each catchment finger may further comprise a suitable geometry, topography, or other features that may be useful in the collection of the stool sample. For example, a catchment finger may comprise ridges, bumps, “teeth,” pegs, or other topographical features or textures that may aid in the collection of the stool sample. The topographical features may be any suitable geometry (e.g., cylindrical, conical, pyramidal, cuboidal, spheroidal, tetrahedral, polyhedral, etc.). The catchment fingers may comprise multiple geometries, textures, and/or orientations. For example, a catchment finger may comprise stacked or grouped sub-features, e.g., cylinders, prisms, pyramids, spheroids, etc. The arrangement of the sub-features may be organized (e.g., evenly spaced) or randomly arranged. Alternatively or in addition to, the catchment unit may comprise a plurality of geometries. For example, the catchment unit may comprise a brush, bristle, spheres, funnels, cup, bowl, net, mesh, sieve, plate, coils, tubes, plane, etc. In some cases, the catchment unit may take the form of a toilet bowl bristle/brush, a ball, a funnel, coiled wire, suction tube, chain chomp, disposable basin, rollers, dustpan, bulbs, spikes, conveyor belt, mandolin, etc.

In some cases, the mechanical, physical, and/or chemical properties of the stool collector, or a portion thereof (e.g., catchment unit) may be controlled. For instance, it may be desirable to tune one or more mechanical properties (e.g., toughness, elasticity, tensile strength, etc.) of the catchment fingers of the catchment unit. In some cases, the catchment fingers may be flexible and/or deformable. In some instances, it may be desirable to tune a chemical property of the catchment finger. For example, the hydrophobicity of the catchment unit may be adjusted. The physical, mechanical, and/or chemical properties of the stool collector may be modified using a variety or combination of materials and/or coatings to achieve useful properties for collection of the stool sample.

The stool collector may comprise any suitable material may be used for each of the parts of the stool collector. For example, one or more components of the stool collector may comprise a polymer or plastic, including, but not limited to: polyamides, polycarbonate, polyester, polyethylene, polypropylene, polystyrene, polyurethane, polyvinyl chloride, polyvinylidene chloride, acrylonitrile butadiene styrene, polymethyl methacrylate, polytetrafluoroethylene, polyimide, polylactic acid, phenolics, polyetheretherketone, or derivatives thereof (e.g., highly cross-linked, high density, etc.). The stool collector, or portion thereof, may comprise a single polymer type (e.g., a homopolymer) or more than one polymer type (e.g., a copolymer) and comprise a random or arranged organization of monomers. For example, a polymer may be a block polymer, an alternating copolymer, periodic copolymer, statistical copolymer, stereoblock copolymer, gradient copolymers, branched copolymers, graft copolymers, etc. Alternatively or in addition to, one or more components of the stool collector may comprise a metal, e.g., steel, copper, iron, brass, or any other suitable metal. A mixture of materials may be used in the stool collector. For example, the fastening mechanisms may comprise one or more metals, whereas the housing of the one or more chambers may comprise a polymer and/or metal or other material.

The stool sample may be processed to yield a processed stool sample. In some cases, the processing comprises homogenization of the stool sample. The homogenization of the stool sample may be configured to occur automatically or may be initiated by a subject (e.g., via a mobile application that is in communication with a communication interface of the stool collector, or via a switch, button, or other actuating mechanism). The homogenization of the stool sample may comprise mixing, blending, moving, and/or emulsifying the stool sample. In some instances, a fluid or reagent is added to the stool sample and may be incorporated into the stool sample and/or used for homogenization of the stool sample.

The stool collector may comprise or be coupled to a homogenization chamber. The chamber may be a vessel. The chamber may take on any useful shape, e.g., tube, box, cylinder, prism, sphere, flask, etc. The homogenization chamber may be coupled to one or more fluid inlets or fluid flow paths. The stool sample may be directed to and/or deposited into the homogenization chamber (e.g., via retractable catchment fingers). During or following depositing of the stool sample into the homogenization chamber, one or more reagents (e.g., filtered water from the tank, buffers, sample processing solutions, etc.) may be introduced into the homogenization chamber (e.g., via the fluid inlet). The homogenization chamber may comprise elements or units for homogenizing the stool sample. For instance, the homogenization chamber may comprise blades coupled to a motor. The stool sample may be deposited into the homogenization chamber, and a homogenization buffer may be introduced (e.g., filtered water). The stool sample may be homogenization by actuation of the motor coupled to one or more blades to stir, blend, rotate, spin, move, and/or homogenize the sample. In another example, the homogenization chamber may comprise one or more pumps (e.g., a centrifugal pump, displacement pump, rotary pump, piston pump, rotary lobe pump, rotary gear pump, diaphragm pump, screw pump, gear pump, vane pump, peristaltic pump, or a combination thereof) to generate a pressure (e.g. water pressure) to homogenize the sample (e.g., via mixing, vortexing, etc.). The homogenization chamber may comprise a homogenizer, e.g., a rotor stator homogenizer, mechanical mortar, blender, masher, mills (e.g., bead mills), rollers, mixers, and/or beads (e.g., magnetic beads). One or more forces (e.g., centrifugal, gravitational, positive or negative pressure, ultrasonic, magnetic etc.) may be applied to the sample, reagents, and/or other homogenizer materials (e.g., applying a magnetic force to magnetic beads) to homogenize the sample. Alternatively or in addition to, the homogenization chamber may comprise a heating or cooling element, and the sample may be heated or cooled to homogenize the sample.

The homogenization chamber may comprise one or more mechanisms for sealing. For example, the homogenization chamber may comprise one or more doors that may be configured to be closed during a process of stool collection. The one or more doors may comprise hinges, sliding ramps, clamps, gaskets, etc. to close the chamber. In some instances, a sealant or caulking may be used, e.g., a plant resin, wax, tar, gum, clay mortar, lime mortar, putty, chalk, oils, polymers, waxes, etc. The door may comprise desirable material or chemical properties. For example, the door may comprise a hydrophobic material that allows for facile cleaning and/or sterilization of the unit.

The plurality of sensors may comprise one or more sensors that allow for measuring of a parameter of one or more systems and/or of the subject. In some instances, the sensor may be coupled to the stool collector. In some instances, the sensor may be a part of the stool collector. The sensor may measure a parameter of the stool collector, the stool sample, or the toilet bowl, or a combination thereof. For example, the sensor may comprise a water level sensor, which detects the quantity of water or fluid volume within the toilet bowl, toilet tank, reagent tank, or a fluid flow path connected to the toilet. Alternatively or in addition to, a sensor of the plurality of sensors may measure a parameter of the subject and provide input to the stool collector (e.g., via a feedback loop). For example, the sensor may measure the weight of a subject. Alternatively or in addition to, the stool collector may comprise a weight sensor that detects if a minimum threshold of force (e.g., weight) is applied to the stool collector. In another non-limiting example, a sensor of the plurality of sensors may comprise a motion sensor. A sensor of the plurality of sensors may be configured to detect a change in the environment, e.g., temperature, vibration, light, color, pressure, movement, etc. A combination of sensors may be used, e.g., a weight sensor, a motion sensor, and a water-level sensor. The plurality of sensors may be different from one another (e.g., individual sensors of the plurality of sensors may be configured to perform different functions or operations). In some cases, at least a subset of the plurality of sensors may be the same type of sensor (e.g., temperature or motion sensor).

Collection of the stool sample by the stool collector may be initiated automatically. The stool collector may automatically turn on to collect the stool sample. The stool collector may automatically turn off subsequent to collection of the stool sample. In some instances, an output of one or more of the plurality of sensors may be used to automatically initiate stool collection. For example, the stool collector may be coupled to a toilet seat and comprise a weight sensor. Upon seating of the subject on the toilet seat, the weight sensor may detect a weight or change in weight and direct a signal to the stool collector to turn on the stool collection unit and initiate stool sample collection. Alternatively or in addition to, a plurality of sensors may be used to automatically turn on the stool collector, or portion thereof, and/or initiate stool sample collection. For example, the stool collector may comprise a sensor that detects motion, vibration, light, color, etc. Upon detection of a change in the environment (e.g., weight, temperature, vibration, etc.), the sensor may direct a signal to the stool collector to automatically initiate stool sample collection. Initiation of stool collection may comprise extension of the catchment unit to collect the stool sample. Following stool sample collection, the stool collector may direct the stool sample to the sample homogenization chamber, e.g., via a fluid path (e.g., a pipe, tube, funnel), or via retraction of the catchment unit to deposit the sample into the homogenization chamber.

One or more processes of stool collection described herein may be performed automatically or manually. In some cases, collection of the stool sample by the stool collector may be initiated manually (e.g., via an input from the subject). The stool collector may comprise a communication interface that can communicate with an external device, e.g., a mobile device, laptop, computer, cell phone, tablet, etc. The subject may interact with the external device (e.g., via a mobile application, a software application, etc.), thereby turning on the stool collector. Alternatively or in addition to, the stool collector may comprise buttons, switches, or other mechanical actuators. The subject may interact with the stool collector to manually turn the stool collector on and/or off. In some cases, the stool collector may automatically turn off subsequent to collection of the stool sample. The stool collector may optionally or in addition to be turned off manually. Initiation of stool collection may comprise extension of the catchment unit to collect the stool sample. Following stool sample collection, the stool collector may direct the stool sample to the sample homogenization chamber, e.g., via a fluid path (e.g., a pipe, funnel), or via retraction of the catchment unit to deposit the sample into the homogenization chamber.

The stool collector may comprise a source of reagents that couples to the fluid flow path. The reagents may be a solid, liquid, gas or a combination thereof. The reagents may be used for the collection and/or processing of the stool sample. In some instances, one or more reagent comprises water. The water may be retrieved from the tank of the toilet. Alternatively or in addition to, the water may be retrieved from a source that supplies water to the toilet (e.g., external pumps, external source of water, pipes). In some instances, one or more reagent comprises a solution. The solution may comprise a buffer. The buffer may be used for sample homogenization and may be selected based on the desired properties. For example, the buffer may be used for homogenization of the stool sample and may comprise cell-compatible buffers (e.g., phosphate buffered saline (PBS), 4-(2-hydroxyethyl)-1-piperazineethanesulfonic acid (HEPES), Tris-buffered solution, Tween, lysogeny broth etc.). In some instances, one or more reagents may be used for sterilization of a part of the system (e.g., the stool collector, toilet bowl, etc.). In such cases, the sterilization reagents may comprise any suitable sterilization reagent, e.g., bleach, chlorine, antimicrobial agents, alcohol, an acid, a base, minerals, soap, etc. or a combination thereof. In cases where the reagent comprises a solid, a buffer may be added to reconstitute and/or dilute the reagents to an appropriate working concentration.

The stool collector may be coupled to and/or comprise one or more fluid inlets and outlets. A fluid inlet may be used to introduce one or more reagents to a portion of the stool collector (e.g., the homogenization chamber). For example, the fluid inlet may be used to introduce water, a buffer, or other reagent into the portion of the stool collector. The one or more reagents may be introduced to the stool collector via a coupled fluid flow path. The fluid inlet of the stool collector or portion thereof (e.g., homogenization chamber) may be coupled to a fluid flow path, e.g. via a hose, tube, pipe, funnel, etc. The fluid inlet may be coupled to or in fluid communication with a source of reagents (e.g., water, buffers, sterilization solutions, etc.). The fluid inlet may comprise one or more valves (e.g., y-valve, y-tube, T-valve, etc.) that may control the flow volume or rate of the reagents into the portion of the stool collector. In some instances, the stool collector is coupled to and/or further comprises a fluid outlet, which may be used to direct the stool sample or processed stool sample away from the stool collector and/or homogenization chamber to be collected. The sample outlet may be used to direct the stool sample to a collection unit, e.g., collection chamber.

In some instances, the fluid flow path may comprise a plurality of valves that may be used to control the flow volume, flow direction, or rate of a fluid into or out of the stool collector, or portion thereof. In some cases, it may be desirable to have a valve that can interface with more than one inlet or outlet or portions of the fluid flow path. In such cases, a custom-built valve may be used. For example, a 3-way luer valve may be used. The 3-way luer valve may interface with a custom-built base. The custom-built base may accommodate one or more gears which may interface with the valves (e.g., 3-way luer valve) and another part, e.g., a motor. Use of such a custom-built valve may allow for programmable and optionally automated 3-way valving, obviating the need for solenoids, which may be electricity-inefficient, susceptible to overheating, and prone to leakage.

In some instances, the stool collector comprises a filter for filtering the water. The filter may be used to purify the water and/or remove contaminants such as dirt, debris, ions, biological particles (e.g., cells, nucleic acid molecules, lipids, carbohydrates, proteins, minerals, heavy metals, etc.). The filter may be used to change one or more qualities of the water, e.g., pH, temperature, ion concentration. The filter may additionally comprise a heating or cooling element. The filter may comprise an activated carbon filter, a reverse osmosis filter, an alkaline or acid ionizer, a UV filter, an infrared filter, a size exclusion filter, an X-ray or gamma ray filter, or any combination thereof.

The reagents may be stored in a reservoir and directed to the fluid flow path using a variety of approaches. The reagents may be directed using, for example, one or more pumps (e.g., mechanical pumps). In some cases, the system may comprise a pump connected to the fluid flow path and/or the source of reagents. The pump may be configured to direct the reagents to the fluid flow path. The pump may be a rotary pump, gear pump, piston pump, diaphragm pump, screw pump, vane pump, peristaltic pump, centrifugal pump, etc., or variations or a combination thereof. In some cases, the reagents may be directed to the fluid flow path using an applied or generated force, e.g., hydraulic, centrifugal, gravitational, frictional, tensional, spring, pneumatic, etc. One or more parts may be coupled to a power supply. Alternatively or in addition to, one or more parts may be configured to couple to an electrical outlet.

Following collection of the stool sample, further processing may be performed. In some cases, the processing comprises sample homogenization, as described herein. The homogenized and/or processed stool sample may be directed along a fluid flow path to yield a processed stool sample and may be collected. In some cases, the processed sample is directed to a fluid outlet of the homogenization chamber and to a collection unit (e.g., chamber). The collection unit may be a vessel, e.g., tube, container, box, bag, device, etc.

Following collection of the stool sample, the stool collector, or a portion thereof, may be cleaned and/or sterilized automatically. In some instances, cleaning comprises introduction of a sterilization reagent (e.g., bleach, chlorine, antimicrobials, etc.) to one or more parts of the stool collector. In some cases, the cleaning reagent may be introduced to the stool collector, or portion thereof, through one of the fluid inlets. In some instances, a pump may be used to direct the sterilization reagent to the stool collector, or portion thereof. In some cases, the sterilization reagent may be used to clean the stool collector, or portion thereof, and the toilet bowl, or portion thereof. In some cases, the sterilization reagent may be introduced to the stool collector, or portion thereof, via spraying, pressure, vapor deposition, pumping, flooding, immersion, or other dispersion technique. The sterilization of the stool collector, or portion thereof, may occur automatically. For example, following stool collection, the stool sample may be deposited into a chamber (e.g., for homogenization and/or stool processing). Following the deposit of the stool sample, the sterilization may be initiated (e.g., via feedback control system). Alternatively or in addition to, a sensor may be used to detect when sterilization is needed. For example, an optical sensor may be used to detect stool specimens on the stool collector or portion thereof, thereby initiating sterilization. In another example, a weight sensor (e.g., on the catchment unit) may be used to provide an input for a sterilization control mechanism. Alternatively or in addition to, a timer may be integrated into the stool collector, such that sterilization is programmed to occur at a given or programmed frequency (e.g., once per minute, once per hour, once per day). In some cases, the subject may be able to initiate the sterilization. In some cases, the sterilization may be manually performed.

The stool collector may be coupled to a fluid flow path. The fluid flow path may be separate from the stool collector. The fluid flow path may be connected and/or coupled to the stool collector (e.g., via an inlet or outlet).The fluid flow path may be directed towards the stool collector, away from the stool collector, or both. For example, the stool collector may comprise an inlet and/or outlet which allows entrance of a reagent (e.g., water, a buffer, etc.) into a portion of the stool collector (e.g., chamber). The stool collector may comprise an inlet and/or outlet which allows exit of a material (e.g., stool sample, processed stool sample) out of the stool collector. In some cases, the stool sample may be directed along a fluid flow path (e.g., homogenization chamber) to process the stool sample and/or collect the stool sample.

The stool collector may comprise a communication interface that allows for transmitting and/or receiving data from a portion of the stool collector (e.g., from a sensor). In some cases, the data may be transmitted to an electronic device in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The stool collector may comprise a port for communication and/or a power supply (e.g., universal serial bus (USB), USB-type C, Thunderbolt, etc.).

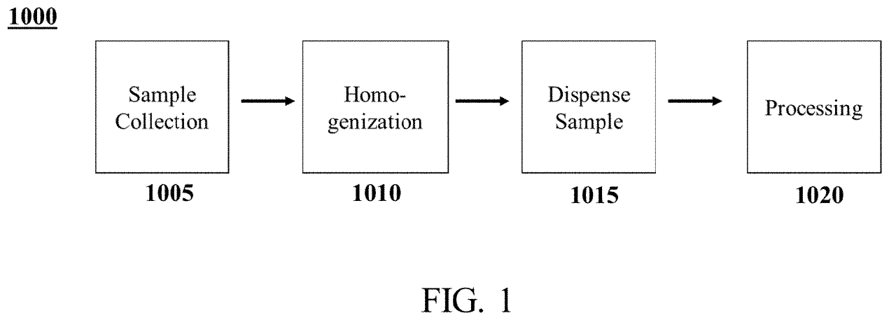

FIG. 1 schematically illustrates an example workflow 1000 for processing a stool sample. In process 1005, a stool sample may be collected. In process 1010, the stool sample may be processed, e.g., homogenized. Process 1010 may comprise one or more operations. For example, the stool sample may be directed to a homogenization chamber, which may be coupled to a fluid flow path comprising homogenization reagents (e.g., filtered water, buffers, salts, etc.). The stool sample and/or homogenization reagents may be homogenized using a variety of approaches (e.g., vortexing, mixing with high water pressure, etc.), as described herein, to yield a processed stool sample. In process 1015, the processed stool sample is directed to a further processing unit. In process 1020, further processing of the processed stool sample may be performed. For example, the stool sample may be analyzed. For example, one or more biological particles of the stool sample (e.g., a cell, a nucleic acid molecule, a protein, a lipid, a carbohydrate, or a combination thereof) may be further processed and analyzed (e.g., via sequencing, spectrophotometry, PCR, mass spectrometry, immunoassay, Raman spectroscopy, etc.).

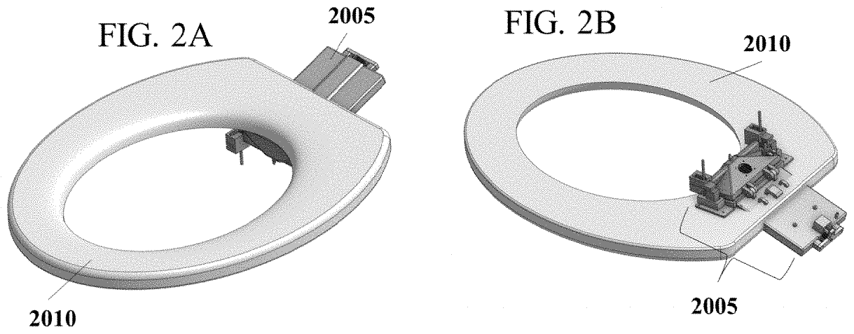

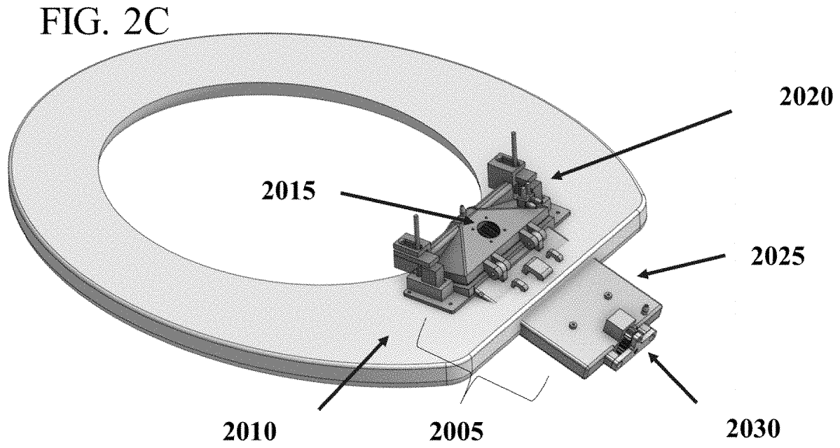

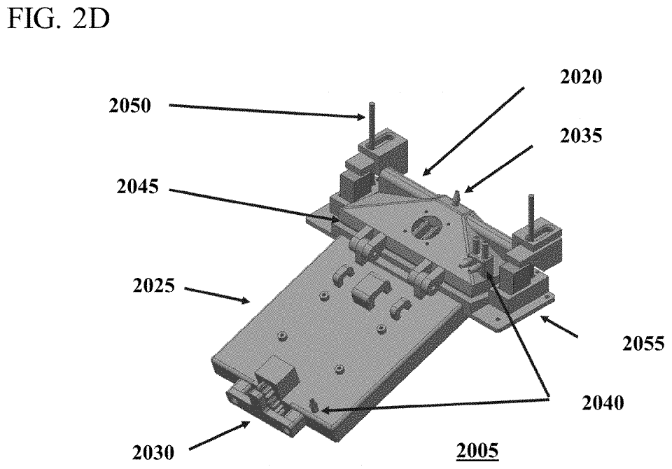

FIG. 2 schematically shows an example stool collector. FIG. 2A shows a stool collector 2005 coupled to a toilet seat 2010. FIG. 2B shows the underside of the toilet seat 2010 and the coupled stool collector 2005. FIG. 2C shows an enlarged view of the underside of the toilet seat 2010 and the stool collector 2005. In some instances, the toilet seat 2010 may be custom built with the stool collector 2005. In other instances, the stool collector 2005 may be configured to couple to a commercially available toilet seat. The stool collector 2005 may comprise a plurality of parts such as one or more motors 2015, an opening and closing mechanism 2020, a chamber 2025 which may house the catchment unit, and/or gears 2030. The one or more motors 2015 may be used to homogenize and/or process the stool sample. The opening and closing mechanism 2020 may comprise a hinge, a sealing mechanism, and/or additional motors, which may facilitate sealing of the stool collector 2005, or portion thereof (e.g., a homogenization chamber). Chamber 2025 may house the catchment unit, which may comprise a catchment arm comprising catchment fingers, which may extend towards the toilet bowl for stool sample collection. Chamber 2025 may also comprise the homogenization chamber. Alternatively or in addition to, the homogenization chamber and chamber 2025 may be the same. The gears 2030 may be configured to interface with another motor (not shown) and chamber 2025 to extend and/or retract the catchment unit.

FIG. 2D shows another view of the example stool collector. In addition to the opening and closing mechanism 2020, the chamber 2025 housing the catchment unit, and gears 2030, the stool collector 2005 may further comprise one more fluid inlets and/or outlets 2035. The fluid inlet and/or outlets 2035 may allow for introduction of reagents (e.g., homogenization buffer) into the stool collector 2005 or portion thereof (e.g., the homogenization chamber). The fluid inlet and outlet may also allow for directing of the stool sample (e.g., after processing) away from the stool collector and to a collection unit (not shown). The stool collector 2005 may also comprise drain connectors 2040, which may prevent overflow of the chamber 2025. The stool collector 2005 and/or the chamber 2025 may comprise mechanisms for sealing the chamber 2025. In some instances the chamber 2025 may comprise a hinged opening and closing mechanism 2020 which may be driven by a plurality of motors (not shown). The chamber 2025 may additionally comprise a rubber gasket 2045 that may be used to seal the chamber 2025. The stool collector 2005 may comprise guide screws 2050 for sealing the motors and a mounting mechanism 2055, to secure the stool collector 2005 to the toilet seat 2010. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

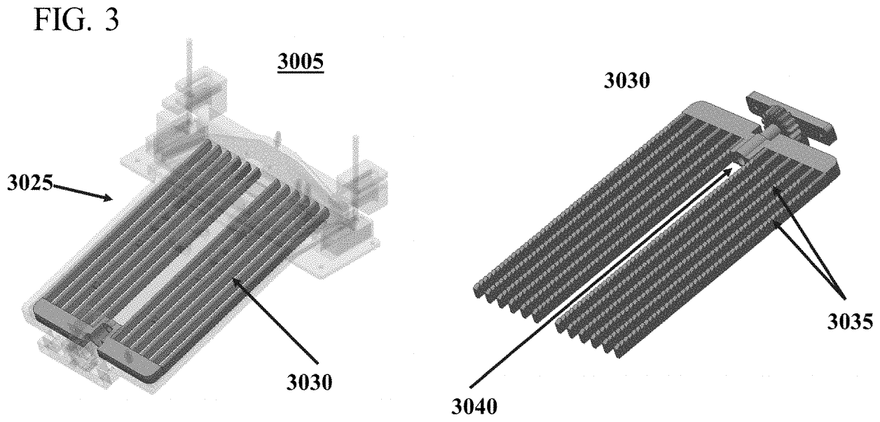

FIG. 3 schematically shows an example catchment unit. The stool collector 3005, which may be substantially similar to 2005, may comprise the catchment unit 3030 within the chamber 3025, which may be substantially similar to 2025. The catchment unit 3030 may comprise a plurality of parallel catchment fingers 3035 or any other useful form or geometry. The catchment fingers 3035 may comprise textures or topographical features, e.g., “teeth”, bumps, ridges, etc. The catchment unit 3030 may also comprise a gear set 3040, which may be coupled to a motor (not shown). The gear set 3040 and motor may be used to extend and/or retract the catchment unit 3030. A guide (not shown) may be used to direct catchment fingers in specific orientations, e.g., fan, half-moon.

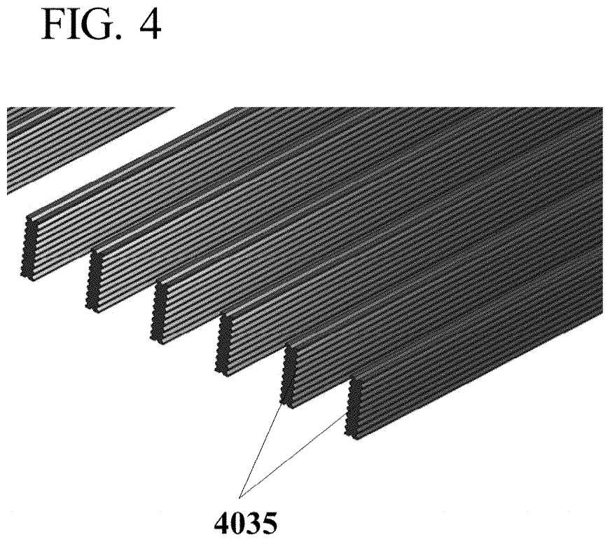

FIG. 4 schematically shows an example geometry of the catchment fingers of a catchment unit. The catchment fingers 4035, which may be substantially similar to 3035, may comprise a plurality of materials, textures, and orientations. For instance, the catchment fingers 4035 may comprise a plurality of stacked cylinders, cubes, pyramids, or any other useful shape or geometry.

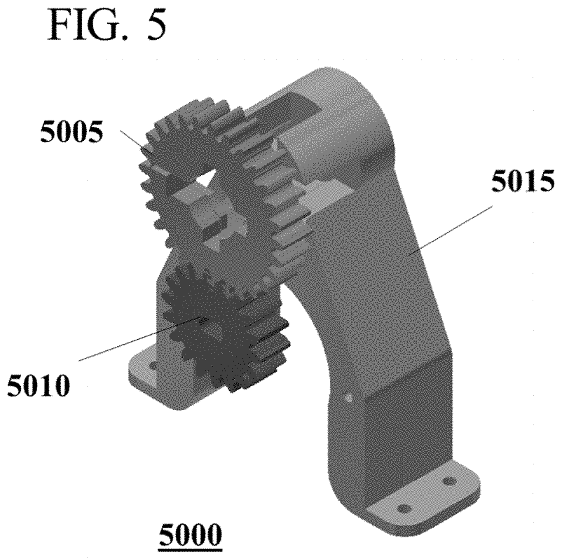

FIG. 5 schematically shows an example valve that may be used in one or more methods or systems described herein. The valve may comprise a housing 5000, which may house the luer valve (not shown), which may allow for programmable, automated valving for a plurality of inlets and outlets. For example, the luer valve and connections thereto may allow for valving of 1, 2, 3, 4, 5, 6, 7, 8, 9, 10 or more inlet and outlets. In one example, the luer valve allows for valving of 3 inlets and outlets. The luer valve may interact with a first gear 5005 that may be configured to interface with a commercially available 3-way luer valve. A second gear 5010 may be configured to interface with a motor (e.g., stepper motor). The first gear 5005 and the second gear 5010 may be secured to a custom-built base 5015. The custom-built base 5015 may assume a variety of useful geometries and can be modulated. For example, the custom-built base may be machined, 3D printed, injection molded, etc.

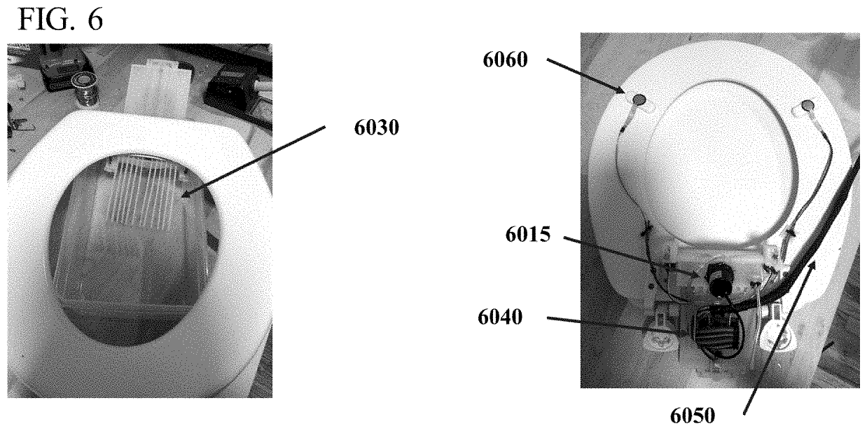

FIG. 6 shows an example of an assembled stool collector. The stool collector may comprise a catchment unit 6030 which comprises parallel catchment fingers. A plurality of sensors 6060 (e.g., weight sensors) may be coupled to the toilet seat. The stool collector may comprise a homogenization motor 6015, which may be coupled to an electronics and motor unit 6040. External tubing 6050 and/or wiring may also be integrated with the stool collector.

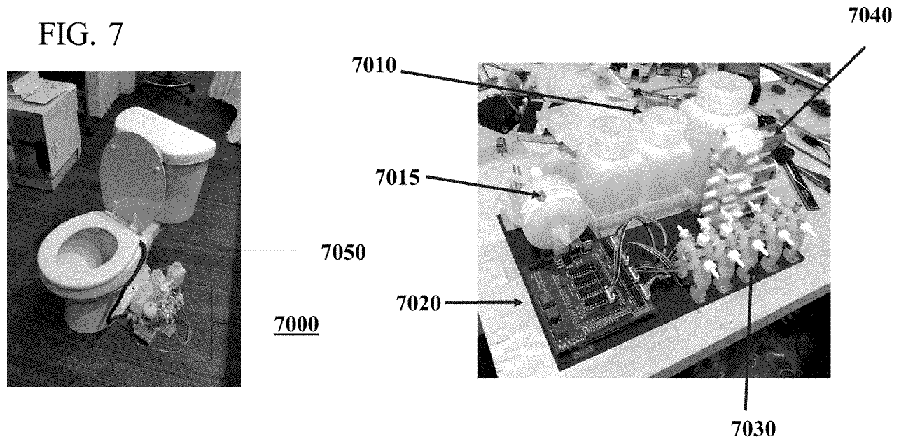

FIG. 7 shows an example of a source of reagents, pumps, and valves that is coupled to the fluid flow path. The stool collector may be coupled to a fluid flow path that may be coupled to the source of reagents 7000, e.g., via external tubing 7050 and/or wiring, which may be substantially similar to 6050. The external source of reagents 7000 may comprise a plurality of parts, e.g., a filter 7015 that may filter water or another reagent. The water that feeds into the filter may be taken from the toilet tank (not shown) or another source (e.g. water supply that feeds into the toilet). The source of reagents 7000 may comprise a plurality of reservoirs 7010 comprising reagents. For example, one of the reservoirs may comprise water, a cleaning reagent, and/or other buffers. Another reservoir of the plurality of reservoirs may comprise the same reagent or a different reagent, or a combination of reagents. For example, one reservoir may comprise water, another reservoir may comprise bleach or other sterilization solution, and yet another reservoir may comprise a buffer. Alternatively, or in addition to two reservoirs may comprise water and another additional reservoir may comprise a cleaning solution. Any number of reservoirs may be used that may be useful. For example, the source of reagents 7000 may comprise 1, 2, 3, 4, 5, 6, 7, 8, 9, 10, 20, 30 or more reservoirs. The source of reagents 7000 may further comprise one or more pumps 7040, valves 7030 (e.g., 3-way valves), a control board 7020, or a combination thereof.

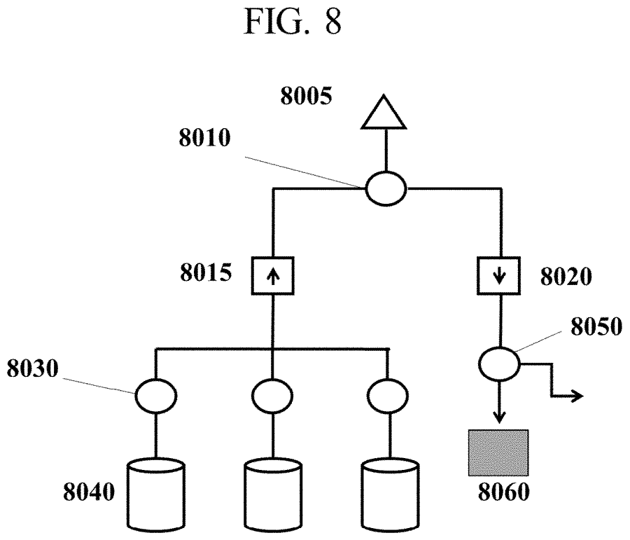

FIG. 8 shows an example process flow diagram of the source of reagents coupled to the stool collector and collection of the stool sample. A fluid inlet and/or outlet 8005, which may be substantially similar to 2035, may be coupled to the stool collector. The fluid inlet and/or outlet 8005 may be connected to valve 8010, such as an automated 3-way luer valve. Valve 8010 may be connected to pumps 8015 and 8020. Pumps 8015 and 8020 may be diaphragm pumps or any other suitable pump. Pump 8015 may be connected to valve 8030, which may be connected to reservoir 8040. Pump 8015 may be connected to a plurality of valves, which may each be connected to a separate reservoir. Reservoir 8040 may comprise useful reagents, e.g., water, sterilization agents, buffers (e.g., homogenization buffer), etc. In some cases, water may be directed from an external source (e.g., pipe or toilet tank) and may be filtered and used for dilution of the reagents in each or some of the reservoirs. Pump 8015 may be configured to direct a reagent from reservoir 8040 to the fluid inlet and/or outlet 8005 and to the stool collector. Further, pump 8020 may be connected to valve 8050, which may be connected to a waste stream and/or a sample collection unit 8060, which may comprise a vessel or container for collection of the stool sample. Pump 8020 may be configured to direct the stool sample (e.g., processed and/or homogenized stool sample) to the waste stream or collection unit 8060.

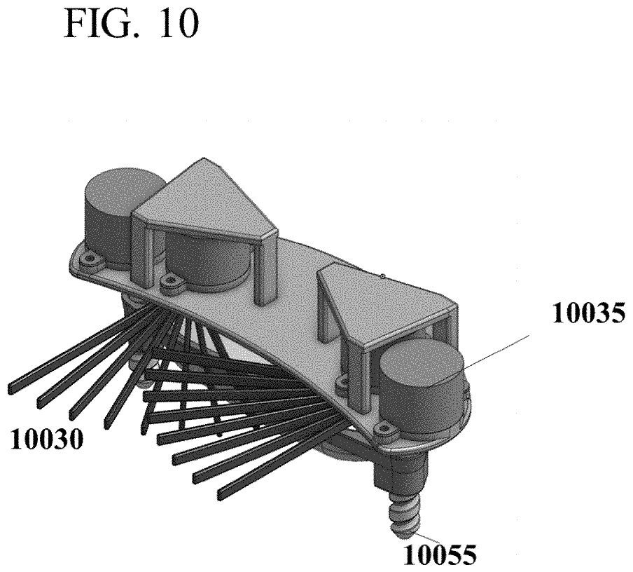

FIG. 10 shows another embodiment of a stool collector. The stool collector may comprise one or more catchment units 10030. In such an example, the stool collector comprises two catchment units 10030. Each catchment unit comprises a plurality of catchment fingers that are arranged in a fan shape and collapse upon retraction. A single or both catchment units may be used. In some cases, both catchment units are used and the catchment units may overlap across an area. The stool collector may also comprise one or more parts useful for stool sample collection (See, e.g., FIG. 2D). For example, the stool collector may comprise a chamber housing the catchment units, gears, the stool one more fluid inlets and/or outlets, which may be coupled to a reagent source. The stool collector may also comprise drain connectors, which may prevent overflow of the chamber. The stool collector may comprise mechanisms for sealing the chamber. The chamber may comprise a hinged opening and closing mechanism which may be driven by a plurality of motors 10035. The chamber additionally may comprise sealing mechanisms (e.g., sealants, gaskets, etc.) that may be used to seal the chamber. The stool collector may comprise guide screws for sealing the motors and a mounting mechanism 10055, to secure the stool collector to the toilet. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

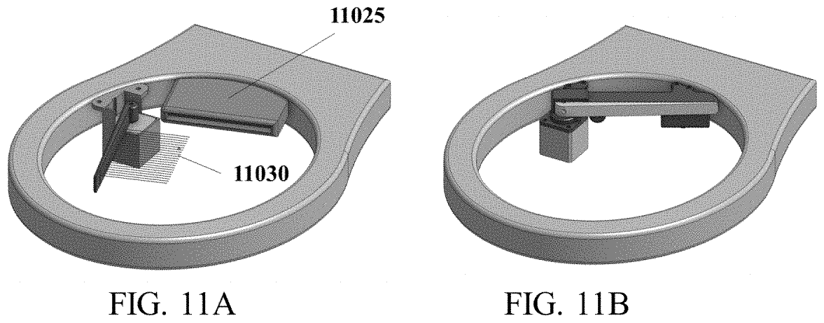

FIG. 11 shows another embodiment of a stool collector. The stool collector may comprise one or more catchment units 11030. The catchment unit 11030 may comprise a plurality of catchment fingers that are arranged in in parallel. It will be appreciated that the catchment fingers may be arranged in any useful order or shape (e.g., fan, circular, parallel, etc.). The catchment unit may be configured to swivel away from the housing chamber 11025, e.g., for stool sample collection. For example, the catchment unit 11030 may be hinged to a separate part of the toilet seat. The catchment unit 11030 may be configured to fit into the housing chamber 11025, which, in some instances, also functions as the homogenization chamber. In some cases, following stool sample collection, the catchment unit may deliver the sample to the housing chamber 11025. Additionally, the stool collector may also comprise one or more parts useful for stool sample collection (See, e.g., FIG. 2D). For example, the stool collector may comprise a chamber housing the catchment units, gears, one or more fluid inlets and/or outlets, which may be coupled to a reagent source. The stool collector may also comprise drain connectors, which may prevent overflow of the chamber. The stool collector may comprise mechanisms for sealing the chamber. The chamber may comprise a hinged opening and closing mechanism which may be driven by a plurality of motors. The chamber may optionally comprise sealing mechanisms (e.g., sealants, gaskets, etc.) that may be used to seal the chamber. The stool collector may comprise guide screws for sealing the motors and a mounting mechanism 10055, to secure the stool collector to the toilet. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

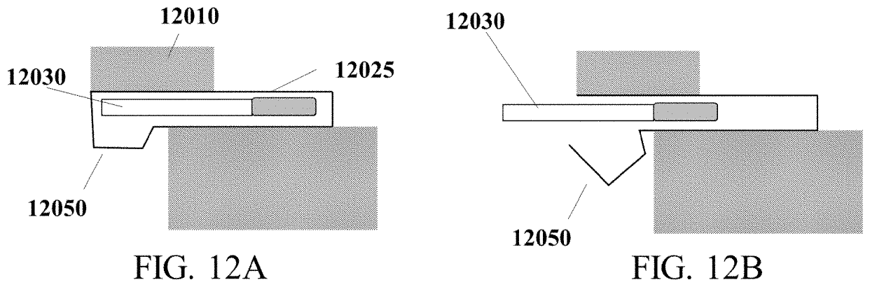

FIG. 12 shows another embodiment of a stool collector. FIG. 12A shows the stool collector in a closed configuration. The stool collector may be integrated with a toilet seat 12010, which may be substantially similar to 2010. The stool collector may comprise one or more catchment units 12030. The catchment unit 12030 may be housed in a chamber 12025, which may be substantially similar to 2025. Chamber 12025 may comprise a movable unit 12050 that allows for the catchment unit 12030 to extend towards the toilet bowl. The movable unit 12050 may be a door, a sliding compartment, etc. FIG. 12B shows the stool collector in an open configuration, e.g., during sample collection. The movable unit 12050 may be opened to allow extension of the catchment unit 12030 towards the toilet bowl. Following collection of the stool sample, the catchment unit may return to the configuration show in FIG. 12A. Additionally, the stool collector may also comprise one or more parts useful for stool sample collection (See, e.g., FIG. 2D). For example, the stool collector may comprise one more fluid inlets and/or outlets, which may be coupled to a reagent source. The stool collector may also comprise drain connectors, which may prevent overflow of the chamber. The stool collector may comprise mechanisms for sealing the chamber. The chamber may comprise a hinged opening and closing mechanism which may be driven by a plurality of motors. The chamber may optionally comprise sealing mechanisms (e.g., sealants, gaskets, etc.) that may be used to seal the chamber. The stool collector may comprise guide screws for sealing the motors and a mounting mechanism, to secure the stool collector to the toilet. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

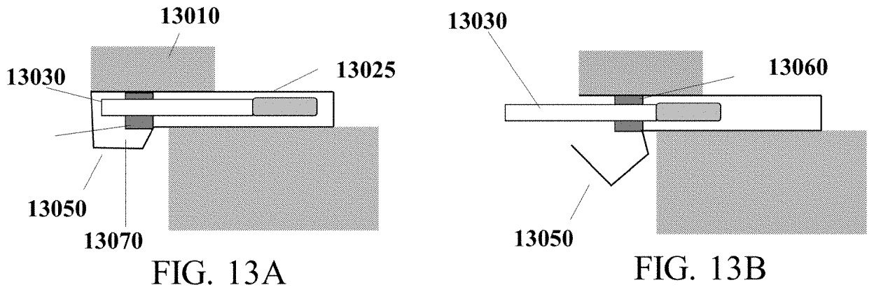

FIG. 13 shows another embodiment of a stool collector comprising a gasket for sample collection. FIG. 13A shows the stool collector in a closed configuration. The stool collector may be integrated with a toilet seat 13010, which may be substantially similar to 2010 and 12010. The stool collector may comprise one or more catchment units 13030. The catchment unit 13030 may be housed in a chamber 13025, which may be substantially similar to 2025 and 12025. Chamber 13025 may comprise a movable unit 13050 that allows for the catchment unit 13030 to extend towards the toilet bowl. The movable unit 13050 may be a door, a sliding compartment, etc. The stool collector may comprise a stopper 13060, e.g., a rubber gasket, which may serve to separate a second chamber 13070 from the chamber 13025. FIG. 13B shows the stool collector in an open configuration, e.g., during sample collection. The movable unit 13050 may be opened to allow extension of the catchment unit 13030 towards the toilet bowl. Following collection of the stool sample, the catchment unit may return to the configuration show in FIG. 13A. The stopper 13060 may be used to separate the chambers 13025 and 13070. In some instances, the stopper 13060 may also be used for depositing the sample into chamber 13070, which may then be used for homogenization of the sample. Additionally, the stool collector may also comprise one or more parts useful for stool sample collection (See, e.g., FIG. 2D). For example, the stool collector may comprise one more fluid inlets and/or outlets, which may be coupled to a reagent source. The stool collector may also comprise drain connectors, which may prevent overflow of the chamber. The stool collector may comprise mechanisms for sealing the chamber. The chamber may comprise a hinged opening and closing mechanism which may be driven by a plurality of motors. The chamber may optionally comprise sealing mechanisms (e.g., sealants, gaskets, etc.) that may be used to seal the chamber. The stool collector may comprise guide screws for sealing the motors and a mounting mechanism, to secure the stool collector to the toilet. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

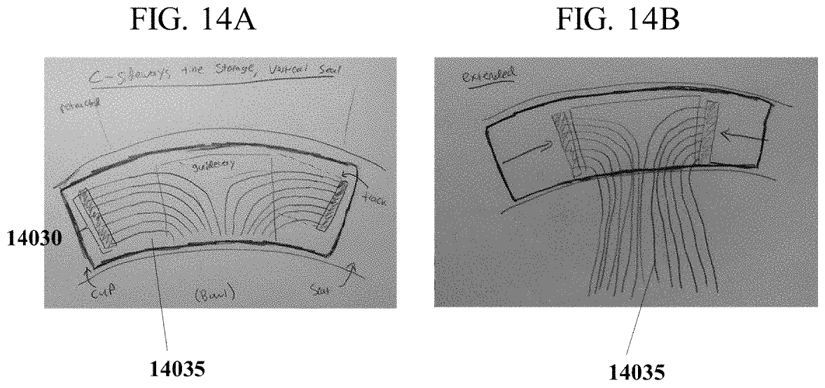

FIG. 14 shows another embodiment of a stool collector. FIG. 14A shows the stool collector in a closed configuration. The stool collector may comprise one or more catchment units 14030. The catchment unit 14030 may comprise a plurality of catchment fingers 14035. The stool collector may comprise units that move the catchment units 14030 toward one another. FIG. 14B shows the stool collector in an open configuration, e.g., during sample collection. The catchment units 14030 may be moved towards one another (e.g., via a gear coupled to screws, a motor, etc.). The catchment fingers 14035 may bend along a path towards the toilet bowl. Following collection of the stool sample, the catchment unit may return to the configuration show in FIG. 14A. Additionally, the stool collector may also comprise one or more parts useful for stool sample collection (See, e.g., FIG. 2D). For example, the stool collector may comprise one more fluid inlets and/or outlets, which may be coupled to a reagent source. The stool collector may also comprise drain connectors, which may prevent overflow of the chamber. The stool collector may comprise mechanisms for sealing the chamber. The chamber may comprise a hinged opening and closing mechanism which may be driven by a plurality of motors. The chamber may optionally comprise sealing mechanisms (e.g., sealants, gaskets, etc.) that may be used to seal the chamber. The stool collector may comprise guide screws for sealing the motors and a mounting mechanism, to secure the stool collector to the toilet. In some cases, the mounting mechanism may comprise a mounting bracket. In other cases, the mounting mechanism may comprise other coupling mechanisms such as complementary threads, snap-fit joints, adhesives, or other interlocking mechanisms e.g., hook and loop, latches, snap-ons, buttons, nuts and bolts, screws, magnets, form-fitting pairs, hooks and loops, latches, threads, screws, staples, clips, clamps, prongs, rings, brads, rubber bands, rivets, grommets, pins, ties, snaps, Velcro, vacuum, seals, or a combination thereof.

### Computer Systems

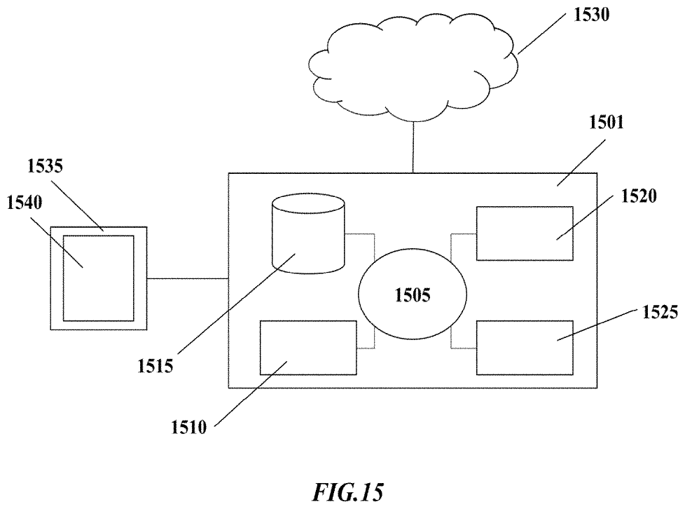

The present disclosure provides computer systems that are programmed to implement methods of the disclosure. FIG. 15 shows a computer system 1501 that is programmed or otherwise configured to analyze a parameter of stool sample collection. The computer system 1501 can regulate various aspects of sample collection of the present disclosure, such as, for example, monitoring completion of stool sample collection, receiving and/or storing an input from one or more sensors, etc. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, information on stool sample collection. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, determine parameters of stool collection (e.g., time course of sample collection), frequency of collection, analysis of the stool sample, etc.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1—Sterilization of Stool Collector Following Sample Collection

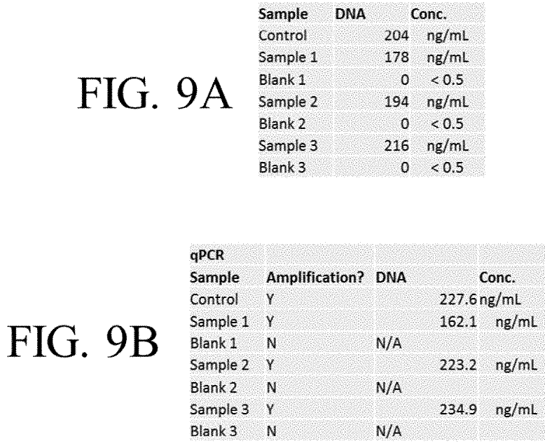

To test the efficacy of the cleaning/sterilization protocol, the amount of DNA detected from a sample can be used as a measurement of the presence of bacteria in a sample. A simulated stool sample that contains Escherichia coli (E. coli) may be collected using the stool collector. The sample may be homogenized and processed by addition of water, and the processed stool sample can be collected. The amount of DNA may be quantified from the collected sample. Following collection of the sample, the sterilization of the stool collector may be performed. A sample of the sterilized collector may be also collected for testing, e.g., quantification of DNA. As shown in FIG. 9, three replicates of samples containing DNA are tested. FIG. 9A shows a table of the measured concentration of DNA, in nanograms per milliliter (ng/mL) of samples containing E. coli and samples collected following the sterilization of the stool collector. Samples 1, 2, and 3 are simulated samples that contain E. coli, and the measurement of the collected samples measure 178, 194, and 216 ng/mL, respectively, of DNA content. The positive control sample has 204 ng/mL of DNA. Blanks 1, 2, and 3, which are samples collected following the sterilization of the collector (i.e., after collection of Samples 1, 2, and 3, respectively), show negligible or non-detectable amounts of DNA, demonstrating that the sterilization of the stool collector may be effective. FIG. 9B shows a table of measured concentration of DNA and detection of amplification products from samples containing E. coli and samples collected following the sterilization of the stool collector. Samples 1, 2, and 3 are simulated samples that contain E. coli, and the measurement of the collected samples measure 162, 223, and 234 ng/mL, respectively of DNA content. The positive control sample has 228 ng/mL of DNA. Blanks 1, 2, and 3, which are samples collected following the sterilization of the collector (i.e., after collection of Samples 1, 2, and 3, respectively), show negligible or non-detectable amounts of DNA. Additionally, all samples were prepared for polymerase chain reaction (PCR). Samples 1, 2, and 3, and the positive control all demonstrated positive amplification (i.e., an amplification product from PCR), whereas Blanks 1, 2, and 3 did not demonstrate amplification (no amplification products). These data show that the sterilization of the stool collector is effective.

What is claimed is:

1. A method for processing a stool sample, comprising:
(a) using a stool collector comprising a plurality of sensors to collect a stool sample of a subject from a location within a toilet bowl, wherein said stool collector is coupled to a fluid flow path separate from said stool collector;
(b) directing said stool sample along said fluid flow path to process said stool sample to yield a processed stool sample; and
(c) collecting said processed stool sample.

2. The method of claim 1, wherein a sensor of said plurality of sensors comprises a non-optical sensor.

3. The method of claim 2, wherein said sensor is a weight sensor.

4. The method of claim 2, wherein said sensor is a water level sensor.

5. The method of claim 2, wherein said sensor is a motion sensor.

6. The method of claim 1, wherein collection of said stool sample by said stool collector is initiated automatically.

7. The method of claim 1, wherein said stool collector automatically turns on to collect said stool sample and automatically turns off subsequent to collection of said stool sample.

8. The method of claim 1, wherein said stool collector comprises a source of reagents that is coupled to said fluid flow path.

9. The method of claim 1, wherein said stool collector is reusable.

10. The method of claim 1, wherein said stool collector is retractable.

11. The method of claim 10, wherein said stool collector deposits said stool sample into a sample homogenization chamber.

12. The method of claim 1, wherein said processing occurs within said location of said toilet bowl.

13. The method of claim 1, further comprising, following (c), automatically cleaning at least a portion of said stool collector.

14. The method of claim 1, wherein said stool collector comprises catchment fingers that collect said stool sample.

15. The method of claim 1, wherein said stool collector comprises electronics coupled to a communication device.

16. The method of claim 1, wherein said fluid flow path is directed out of said toilet bowl.

17. The method of claim 1, wherein said stool collector is attached to a toilet seat coupled to said toilet bowl.

18. The method of claim 1, wherein said stool collector is attached to said toilet bowl.
